(19) United States
(12) Patent Application Publication
(10) Pub. No.: US 2023/0416672 A1
(43) Pub. Date: Dec. 28, 2023

(54) Rhodococcus qingshengii bacterial strain that degrades imazethapyr and stimulates crop growth

(71) Applicant: Organic park limited liability company, Kazan (RU)

(72) Inventors: Anna Yurievna MURATOVA, Kazan (RU); Olga Viktorovna TURKOVSKAYA, Kazan (US)

(73) Assignee: Organic park limited liability company, Kazan (RU)

(21) Appl. No.: 18/038,010

(22) PCT Filed: Nov. 15, 2021
(86) PCT No.: PCT/RU21/50376

§ 371 Date: May 20, 2023

(30) Foreign Application Priority Data
Nov. 27, 2020 (RU) 2020139179

(51) Int. Cl. C12N 1/20; B09C 1/10

(52) U.S. Cl. CPC C12N 1/205; B09C 1/10; C12R 2001/01

(57) ABSTRACT

The proposed disclosure relates to agricultural microbiology, ecology and biotechnology and is intended for remediating soil contaminated with imidazolinone herbicides and stimulating the growth of planted crops. A Rhodococcus qingshengii bacterial strain deposited under VKPM number Ac-2143 was isolated from an enrichment culture based on rhizosphere microflora of soybeans grown on soil treated with the herbicide imazethapyr. The Rhodococcus qingshengii bacterial strain deposited under VKPM number Ac-2143 degrades the herbicide imazethapyr and stimulates the growth of crops planted in soil contaminated with imazethapyr.

### CROSS-REFERENCE TO RELATED APPLICATIONS

This application is a U.S. national stage application of an international application PCT/RU2021/050376 filed on 15 Nov. 2021, published as WO/2022/115002, which international application claims priority of a Russian Federation patent application RU2020139179 filed on 27 Nov. 2020.

### FIELD OF THE INVENTION

The invention relates to the field of agricultural microbiology, ecology and biotechnology and is intended to restore soil from contamination with herbicides of the imidazolinone class, stimulate the growth of planted plants, and is Rhodococcus qingshengii bacterial strain, deposited under VKPM number Ac-2143.

The development of modern agriculture is impossible without the use of fertilizers and chemical plant protection products—pesticides, among which the maximum share is accounted for by herbicides. In the process of use, herbicides enter the soil, where their half-life depends on a large number of factors: the chemical specification of the substance, the type and physical-chemical properties of the soil, and the climatic and geographical features of agricultural areas. Many herbicides (such as imidazolinones) are extremely slow to break down in the soil, and microbial degradation of these persistent compounds may be the only way to restore soil health and ensure new crop yields.

### BACKGROUND OF THE INVENTION

From the current level of development of science and technology, a biological preparation is known that has the property of destroying pesticides using the example of simazine and stimulating soil fertility. The basis of the biological product-biodestructor is a consortium of lactic acid bacteria Streptococcus thermophilus, Streptococcus bovis, Lactobacillus salivarius var. salicinicus, Lactobacillus salivarius var. salivarius, Lactobacillus acidophilus (VKPM-5972), plant polysaccharides, microelements in the form of complexonates, unsaturated fatty acids—precursors of prostaglandins (Pat. RF No. 2203870; Pat. RF No. 2203879).

However, this microbial composition is not capable of decomposing herbicides of the imidazolinone classes and is not capable of stimulating plant growth.

Described is a biological preparation for purification of water, soil, industrial wastewater from pesticides resistant to decomposition, selected from chlorophenoxyacetic acids, such as 2,4-dichlorophenoxyacetic acid (2,4-D), trichlorophenoxyacetic acid (2,4,5-T), chlorophenoxyacetic acid (CPA), phenoxyacetic acid (PAA), -2,4-dichlorophenoxy-α-propionic acid, 2-methyl-4-chlorophenoxy-α-propionic acid, 2,4,5-trichlorophenoxy-α-propionic acid, 2,4-dichlorophenoxy-α-butyric acid, methyl-[1-(butylamino)carbonyl]-1H-benzimidazol-2-ylcarbamate, 2,4-dichlorophenol, imidoclaprid, hexachlorohexane, as well as phenol (Pat RF No. 2484131).

The biological product is an association of bacterial strains Pseudomonas putida VKPM B-10997, Bacillus subtilis VKPM B-10999 and Rhodococcus erythropolis VKPM-Ac-1882 in a mass ratio of (1-2):(1-2):1. The biological product additionally contains a sorbent, organic, mineral and stimulating additives and has plant growth stimulating activity and fungicidal properties.

This biological product has a plant growth-stimulating effect, but is not capable of degrading herbicides of the imidazolinone class.

Known bacterial strain Bacillus megaterium 501, which can be used for the destruction of residual amounts of organophosphorus pesticides in natural environments (AS USSR No. 1735359). However, the indicated microorganism does not exhibit plant growth-stimulating activity.

Also known Rhodococcus wratislaviensis VKM Ac-2623D bacterial strain for cleaning soils contaminated with hexachlorobenzene, lindane, dichlorodiphenyltrichloroethane, dichlorodiphenyldichloroethane, triallate and phthalic acid esters (dibutyl phthalate, dioctyl phthalate) (Pat. RF No. 2562156).

The disadvantage of this invention is the inability of the Rhodococcus wratislaviensis VKM Ac-2623D strain to degrade herbicides of the imidazolinone group.

Known Achromobacter sp. BKM V-2534 D bacterial strain, used for cleaning soils and liquid media, for example, ground and surface waters contaminated with organophosphonates (Pat. RF No. 2401298). The bacterial strain Achromobacter sp. Kg 16 was isolated by enrichment cultures from soil contaminated with glyphosate, deposited in the All-Russian Collection of Microorganisms under the number VKM V-2534 D. The strain utilizes organophosphonates: glyphosate, methylphosphonate, amino-methylphosphonate, phosphonoacetate, 2-aminoethylphosphonate, iminodiacetate. The resistance of the strain to high concentrations of the herbicide glyphosate was shown, which expands the range of its application, including in emergencies.

The indicated strain of Achromobacter sp. BKM B-2534 degrades organophosphonate herbicides, but not imidazolinones, and is unable to stimulate plant growth.

Closest to the proposed microorganism in terms of its ability to biodegrade imazethapyr is a strain of bacteria Pseudomonas sp. IM-4 (Huang et al., 2009). This strain utilizes more than 73% of imazethapyr, which is present as the only source of carbon and energy at a concentration of 50 mg/l in a liquid mineral medium, and is also capable of destroying this herbicide in the soil by 67.6% in 25 days at an initial concentration imazethapyr in soil 10 mg/kg. However, there are no data on the presence of traits in this strain that allow stimulating plant growth (for example, the synthesis of phytohormones, siderophores, and other phytoactive substances), as well as data on stimulating the growth of such cereal crops as wheat or rye.

### OBJECT AND SUMMARY OF THE INVENTION

The task to be solved by the claimed technical solution is to obtain a microbial strain that degrades imazethapyr—an imidazolinone class herbicide—and has the properties of a plant growth stimulator.

The technical result that can be obtained by using the claimed invention is to increase the biodegradation of the herbicide imazethapyr and stimulate plant growth in soil containing its residual concentrations.

The IMZT-32 strain was studied for the presence of properties that directly or indirectly promote plant growth: synthesis of the phytohormone indolyl-3-acetic acid (IAA), fixation of atmospheric nitrogen, dissolution of phosphates, synthesis of the enzyme 1-aminocyclopropane-1-carboxylate (ACC) deaminase, siderophore synthesis, stimulation of germination and development of plant seedlings. It has been established that IMZT-32 is able to produce siderophores, ACC deaminase and IAA phytohormone, stimulate the growth of shoots and roots of common wheat (Triticum aestivum L.) and winter rye (Secale cereale L.) in laboratory vegetative experiments from the 4th day. seedlings.

The strain IMZT-32 was deposited with registration number IBPPM 646 in the Collection of Rhizospheric Microorganisms of the Institute of Biochemistry and Physiology of Plants and Microorganisms of the Russian Academy of Sciences, and then, due to the expansion of the directions of fundamental and applied research, this strain was identified as the species Rhodococcus qingshengii, renamed in Rhodococcus qingshengii OPI-01 and deposited in the All-Russian Collection of Industrial Microorganisms under VKPM number Ac-2143.

The inventive strain is characterized by the following cultural-morphological, physiological-biochemical and biotechnological features.

Morphological signs: In the smears of the daily culture there are short sticks of irregular shape, V-shaped, with patches of weak branching. They are positively colored by Gram. They do not form a dispute. Motionless.

Cultural characteristics: Aerobic. The temperature optimum is 30° C. After incubation on meat-peptone agar in 2 days, it forms rounded, convex colonies with a diameter of about 2 mm with a shiny surface, non-transparent, homogeneous structure and thick oily consistency. Over time, the colonies acquire a pinkish hue. Growth on beveled meat-peptone agar is distinct by a stroke on the 2nd day.

Physiological and biochemical signs: Produces catalase, urease, but not oxidase, lecithinase, lipase, phenylalanyl deaminase. Does not hydrolyze gelatin and starch. When growing on meat-peptone broth, it forms hydrogen sulfide, but not indole and ammonia. Fluorescent and other pigments do not form. Carries out the reduction of nitrates. Able to grow on the environment in the presence of 2.5% NaCl. Utilizes citrate on Simmons medium. In the O/F test, it oxidizes xylose, maltose, but not lactose and mannitol.

Genetic characteristics: Phylogenetic analysis based on the comparison of the nucleotide sequences of the 16SrRNA gene showed that the OPI-01 strain is a representative of the Rhodococcus qingshengii species.

Biotechnological Signs:
the ability to stimulate plant growth, due to the production of the phytohormone IAA, the enzyme ACC-deaminase and siderophores. Synthesizes IAA at a concentration of 8-12 mcg/mL for 7 days cultivation in a medium containing the precursor of the phytohormone amino acid tryptophan. The production of IAA by the strain is determined using HPLC by the appearance and accumulation of the phytohormone. Stimulates the growth of shoots and roots of common wheat (Triticum aestivum L.) and winter rye (Secale cereale L.) in laboratory vegetation experiments from 4 days seedlings.
the ability to degrade the herbicide imazethapyr. Destroys the herbicide imazethapyr at a concentration of 50 mg/L for 7 days by 23% when cultivated in liquid mineral medium MSM. The activity associated with the degradation of the herbicide is determined by the loss of the substrate from the cultivation medium. The residual content of imazethapyr is determined using high performance liquid chromatography (HPLC).

Under laboratory conditions (not in the collection), the culture is stored at 4° C. on columns of agarized (0.6%) LB medium (g): agar, 6; bactotripton, 10; yeast extract—5; NaCl—5; distilled water—1 L; pH 7.0-7.2.

The strain of Rhodococcus qingshengii VKPM Ac-2143 is avirulent (Protocol of an experimental study to determine the average lethal dose (LD50) in white mice, conducted on 16 Apr. 2019-29 Apr. 2019 in the Laboratory of Diagnostic Technologies of the Microbiology Department of the Department of Diagnostics of Infectious Diseases of the Federal State Institution of Higher Education, the Russian Research Anti-Plague Institute “Microbe” of the Federal Service for Supervision of Consumer Rights Protection and Welfare (Federal State Institution of RosNIPCHI “Microbe” of Rospotrebnadzor) and can be used to restore soil from residual contamination with herbicides based on imazethapyr and to stimulate growth plants on such soil.

### PREFERRED EMBODIMENTS OF THE INVENTION

The bacterial strain Rhodococcus qingshengii, deposited under VKPM number Ac-2143, which was initially assigned the laboratory code IMZT-32, was isolated from an enrichment culture based on the microflora of the soybean rhizosphere (Glycine max L.) grown on soil formed botanical herbicide “Fabian”, the active ingredient of which is imazethapyr. To establish an enrichment culture, samples of plant roots with the remaining soil attached to them (no more than 2 mm) were placed in a flask with MSM mineral medium (Huang X., Pan J. J., Liang B., Sun J. Q., Zhao Y. Y., Li S. P. Isolation, characterization of a strain capable of degrading imazethapyr and its use in degradation of the herbicide in soil//Curr. Microbiol.—2009.—Vol. 59.—P. 363-367.) of the following composition (g/L): NH₄NO₃—1.0; K₂HPO₄—1.5; KH₂PO₄—0.5; MgSO₄×7H₂O—0.2; NaCl—0.5. Imazethapyr was added to the medium as the sole source of carbon and energy to a final concentration of 50 mg/L.

Flasks with enrichment cultures were incubated on a shaker at 140 rpm and 30° C. for eight weeks, replacing the medium in the flasks with fresh weekly. At the end of cultivation, tenfold dilutions were prepared from enrichment cultures, which were sown on the surface of MSM agar medium containing 100 mg/L imazethapyr as the sole source of carbon and energy. The inoculations were incubated in a thermostat at 30° C. for 7 days, after which the colonies that appeared were counted and sifted out to obtain pure cultures.

Subsequently, all isolates obtained in this way were tested for the ability to degrade imazethapyr by cultivating them in a liquid MSM medium containing herbicide at a concentration of 50 or 100 mg/L as the only source of carbon and energy. The IMZT-32 strain was distinguished by its ability to degrade the herbicide imazethapyr by 23% in 7 days cultivation.

### INDUSTRIAL APPLICABILITY

The invention is illustrated by the following examples.

### EXAMPLE 1

Biodegradation of imazethapyr and similar in structure metabolites by the strain of Rhodococcus qingshengii VKPM Ac-2143 during cultivation in a liquid mineral medium.

The ability of Rhodococcus qingshengii VKPM Ac-2143 strain to degrade the herbicide imazethapyr and its possible metabolites was studied by cultivating the microorganism in a liquid mineral medium MSM containing imazethapyr at a concentration of 50 mg/L as the only source of carbon and energy or one of the metabolites (2,3-pyridinedicarboxylic acid—PDA or 2-hydroxynicotinic acid—HNA at a concentration of 200 mg/L; Medium pH 6.8-7.0. The inoculation of the microorganism into the medium was carried out until the initial density of the microbial suspension was 0.05 or 0.1 optical units measured at 600 nm. Medium with imazethapyr or metabolites without bacteria was used as a control. All variants of the experiment were repeated three times. Cultivation was carried out for 7 days under aeration on a shaker at 150 rpm, 30° C. At the end of the experiment in the medium after cultivation, the residual content of imazethapyr, PDA and HNA was determined by HPLC (table 1).

TABLE 1
Biodegradation of imazethapyr and closest metabolites - hydroxynicotinic
and pyridinedicarboxylic acids - as the only sources of carbon and
energy in the MSM medium by the Rhodococcus qingshengii
strain VKPM Ac-2143

| | imazethapyr, | HNA, | PDA, |
|---|---|---|---|
| Experience Variant | mg/L | mg/L | mg/L |
| Control | 52.5 ± 1.9 | 219.4 ± 11.6 | 203.6 ± 4.1 |
| (without microorganism) | | | |
| Rhodococcus qingshengii | 40.6 ± 5.6 | 202.1 ± 6.3 | 166.3 ± 8.3 |
| VKPM Ac-2143 | | | |

Analysis of the residual content of the herbicide and metabolites in similar chemical structure in the medium after cultivation for 7 days and in the control variant without the microorganism showed that imazethapyr was destroyed by Rhodococcus qingshengii VKPM Ac-2143 strain by 22.7%, PDA by 18.3%, and HNA by 6.9%.

The data obtained testified to the ability of the Rhodococcus qingshengii VKPM Ac-2143 strain to degrade imazethapyr and metabolites close to it in chemical structure, using them as the only source of carbon and energy.

### EXAMPLE 2

Influence of Rhodococcus qingshengii VKPM Ac-2143 strain on the growth and development of soft wheat plants in soil artificially contaminated with imazethapyr.

The soil in the experiment was artificially polluted with the herbicide preparation “Viaduk VK” (Active substance: imazethapyr, 100 g/L), introducing the drug with water for irrigation at a dosage, according to the instructions, to a final concentration of imazethapyr 10 mg/kg of soil. The soil was moistened to 50% of the total moisture capacity. Seeds of soft wheat (Triticum aestivum L.) varieties “Favorite” 20 pcs. were sown in 1-liter pots containing 1 kg of soil. To stimulate plant growth after germination, the soil was treated twice, on days 5 and 21 of cultivation, with bacteria, introducing a microbial suspension with water for irrigation to a final concentration of the inoculant microorganism in the soil, 107 cells/g. To prepare the suspension, a 3-day-old LB-agar culture of the strain was washed in phos-phate (pH 7.2) buffer and resuspended in water for plant irrigation.

The experiment was carried out under controlled conditions: at a temperature of 24/20° C., a light regime of 14/10 h (illumination—8000 lux), day/night, respectively, maintaining soil moisture at a level of 50% of the total moisture capacity by irrigation with settled tap water, the need for which was determined by weighing vessels. The duration of the experiment was 47 days. At the end of the experiment, the plants were removed from the vessels, the roots were washed from the soil with running water. They were dried with filter paper, the length of roots and shoots was measured, they were separated and dried to constant weight at 65° C. Soil samples (5 g) were taken and air-dried prior to analysis for herbicide. The content of imazethapyr in soil was determined as described previously (Assalin M. R., Queiroz S. C. N., Ferracini V. L., Oliveira T., Vilhena E., Mattos M. L. T. A method for determination of imazapic and imazethapyr residues in soil using an ultrasonic assisted extraction and LC-MS/MS//Bull. Environ. Contam. Toxicol.—2014.—Vol. 93.—P. 360-364) using HPLC.

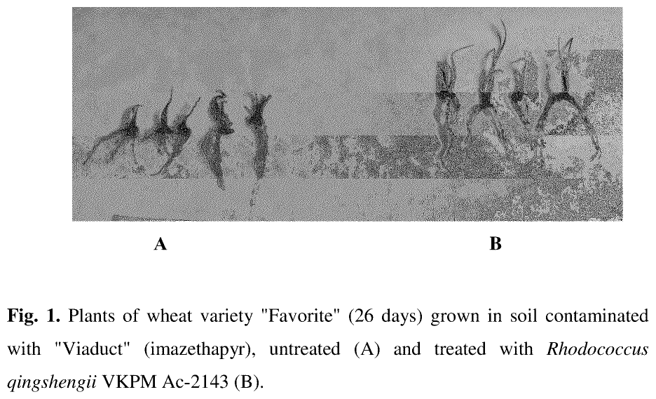

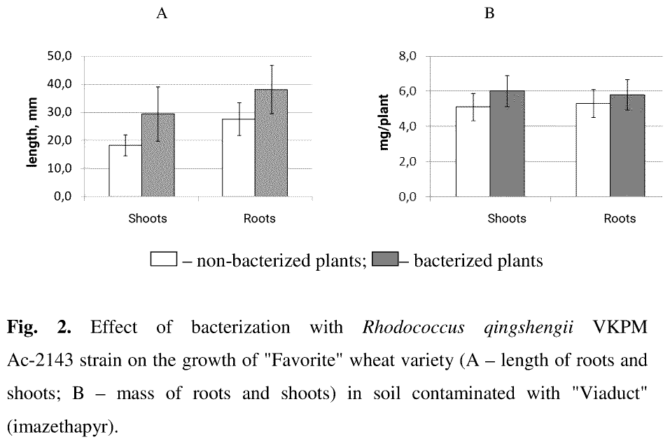

In the soil freshly contaminated with imazethapyr, a strong inhibition of the growth of wheat plants was observed: for 25 days during the growing season, almost all plants in the variant without bacterization remained at the seedling stage. Plants were selected from the untreated and treated by the microorganism soil and compared by morphometric parameters (FIGS. 1 and 2). In bacterized plants, the length of shoots and roots was 61 and 38%, and the increase in biomass was 17 and 9% higher compared to non-bacterized plants, respectively. Thus, in this variant of the experiment, Rhodococcus qingshengii VKPM Ac-2143 strain showed the ability to stimulate plant growth.

The analysis of imazethapyr content in the soil was carried out before sowing plants (starting point) and at the end of the experiment. The results are presented in table 2.

TABLE 2
Changes in the content of imazethapyr
in the soil in a laboratory experiment

| | Imazethapyr | |
|---|---|---|
| Experience Variant | content, mg/kg | Degradation, % |
| The original contaminated soil | 6.17 | |
| Non-bacterized soil, without plants | 5.87 | 4.9 |
| Non-bacterized soil, with wheat | 5.90 | 4.3 |
| Bacterized soil, without plants | 5.36 | 13.1 |
| Bacterized soil, with wheat | 5.78 | 5.7 |

Note. Approximate allowable concentration in the soil for imazethapyr 0.1 mg/kg

Thus, the proposed strain of bacteria has the properties to degrade the herbicide imazethapyr and stimulate the growth of plants grown in soil contaminated with it. The combination of these properties can ensure both the biodegradation of the pollutant in the soil, and the survival and stimulation of the growth of plants planted in polluted soil.

### EXAMPLE 3

Influence of the strain Rhodococcus qingshengii VKPM Ac-2143 on the growth and development of plants of soft spring wheat (Triticum aestivum L.) cv. “Simbirtsit” in sand artificially contaminated with imazethapyr.

“Pivot, VK” (Active substance: imazethapyr, 100 g/l) was used as a model herbicide. Solutions of the drug were applied in the following amounts (per gram of dry quartz sand): 114×10⁻⁷ mL, 28.5×10⁻⁷ mL. The calculation of herbicide concentrations per unit mass of the substrate was carried out based on the bulk density of quartz sand 1750 kg/m³ and an estimated depth of pesticide penetration of 20 cm. The treatment of sand with cells of Rhodococcus qingshengii VKPM Ac-2143 was carried out after the introduction of the herbicide into the soil, at the rate of 10⁷ cells/g of sand. A suspension of microorganism cells was prepared in a sterile Knop solution with trace elements and glycerol as a source of carbon and energy at a concentration of 3 g/L.

Before setting up the experiments, the sand was sterilized by autoclaving at a pressure of 1 atm. within 1 hour. Soil bacterization was carried out in two variants: A—after 1 day after soil treatment with a pesticide, sowing plants—after 14 days, B—after 5 days after adding the pesticide to the sand, sowing plants—after 21 days. Before planting the plants, the sand was moistened with a sterile Knop solution with glycerol at a concentration of 0.5 g/l, but without microelements; after sowing wheat seeds—with a sterile Knop solution without glycerin and trace elements.

Before planting, plant seeds were sterilized with 3% hydrogen peroxide, followed by washing ten times with sterile distilled water.

Plant growth was evaluated 4 weeks after planting. The growth intensity was judged on the basis of morphological parameters (the length of roots and shoots), and the protein content in the roots and shoots of plants was determined by the Lowry method and the chlorophyll content in the leaves was determined by the spectrophotometric method.

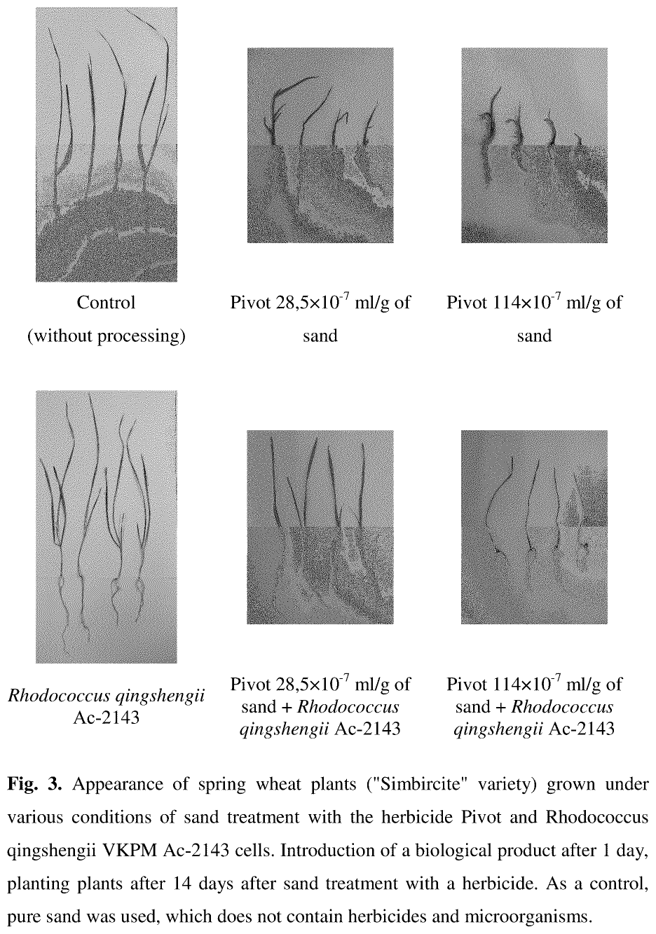

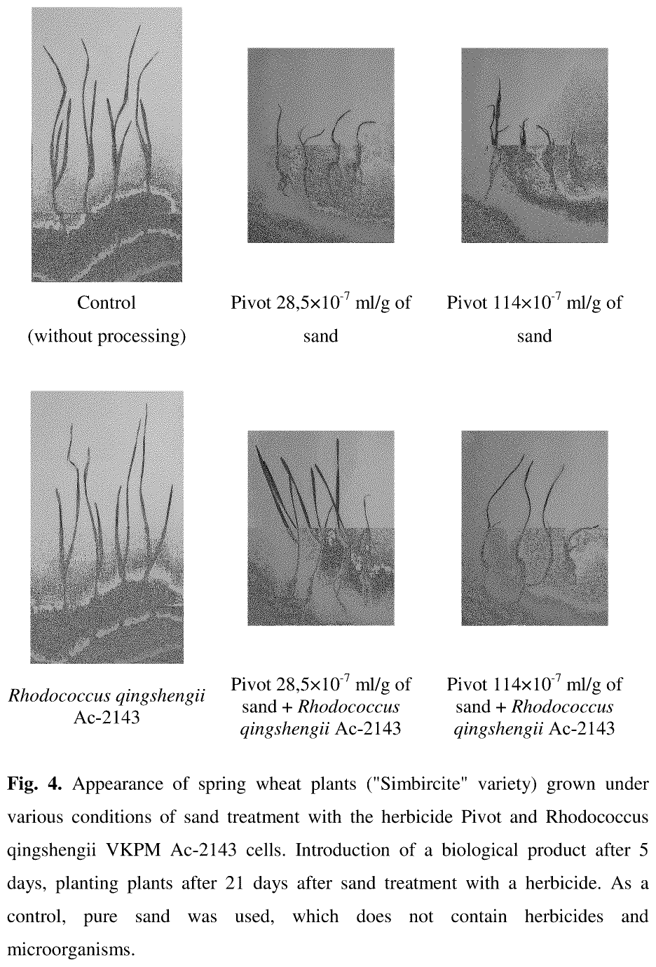

When analyzing the effect of bacterization on the growth of “Simbirtsit” wheat, it was found that the introduction of Rhodococcus qingshengii VKPM Ac-2143 culture into sand has a beneficial effect on the growth and development of plants in contaminated soil (Table 3, FIGS. 3 and 4).

TABLE 3
Morphometric and biochemical parameters of Simbircit wheat plants under the influence
of the herbicide Pivot and the bacterization of Rhodococcus qingshengii VKPM Ac-2143.

| | Processing | | | | | |
|---|---|---|---|---|---|---|
| | Pivot, 0 mL/g | | Pivot, 28.5 × 10⁻⁷ mL/g | | Pivot, 114 × 10⁻⁷ mL/g | |
| | Without | | Without | | Without | |
| Parameter | m/o | Ac-2143 | m/o | Ac-2143 | m/o | Ac-2143 |

A. Introduction of a biological product 1 day after sand treatment with herbicide,
planting plants 14 days after herbicide application
Length, mm

| | | | | | | |
|---|---|---|---|---|---|---|
| root | 121.5 ± 29.1 | 145 ± 33.4 | 47.0 ± 5.6 | 68.1 ± 20.2 | 35.4 ± 9.1 | 42.1 ± 11.3 |
| shoot | 352.8 ± 67.4 | 350.6 ± 45.9 | 99.6 ± 37.4 | 217.1 ± 47.4 | 43.0 ± 17.7 | 119.6 ± 35.1 |

Total protein, % of dry weight

| | | | | | | |
|---|---|---|---|---|---|---|
| root | 1.58 ± 0.11 | 1.34 ± 0.37 | 0.53 ± 0.19 | 1.29 ± 0.35 | 0.29 ± 0.03 | 1.27 ± 0.17 |
| shoot | 0.42 ± 0.22 | 0.19 ± 0.06 | 0.3 ± 0.07 | 0.29 ± 0.05 | 0.18 ± 0.06 | 0.27 ± 0.09 |

Chlorophylls, mcg/g

| | | | | | | |
|---|---|---|---|---|---|---|
| a | 838 ± 301 | 711 ± 257 | 597 ± 277 | 768 ± 192 | 267 ± 54 | 458 ± 116 |
| b | 387 ± 128 | 338 ± 107 | 239 ± 106 | 333 ± 85 | 107 ± 38 | 190 ± 43 |
| general content | 1226 ± 428 | 1049 ± 363 | 836 ± 382 | 1101 ± 276 | 374 ± 92 | 647 ± 159 |

B. Introduction of a biological product 5 days after sand treatment with herbicide,
planting plants 21 days after herbicide application
Length, mm

| | | | | | | |
|---|---|---|---|---|---|---|
| root | 124.4 ± 17.9 | 120 ± 21.2 | 29.5 ± 9.1 | 54.1 ± 12.0 | 28.8 ± 5.5 | 42.8 ± 8.7 |
| shoot | 340.1 ± 36.6 | 353.3 ± 26.5 | 56.4 ± 11.6 | 182.4 ± 25.4 | 31.5 ± 7.8 | 108.0 ± 22.1 |

Total protein, % of dry weight

| | | | | | | |
|---|---|---|---|---|---|---|
| root | 1.26 ± 0.08 | 1.24 ± 0.08 | 0.62 ± 0.04 | 1.49 ± 0.10 | 0.49 ± 0.03 | 1.12 ± 0.15 |
| shoot | 0.51 ± 0.07 | 0.41 ± 0.05 | 0.13 ± 0.01 | 0.28 ± 0.02 | 0.15 ± 0.03 | 0.26 ± 0.03 |

Chlorophylls, mcg/g

| | | | | | | |
|---|---|---|---|---|---|---|
| a | 859 ± 112 | 572 ± 68 | 382 ± 15 | 574 ± 145 | 301 ± 37 | 376 ± 184 |
| b | 377 ± 34 | 284 ± 40 | 170 ± 36 | 286 ± 62 | 154 ± 17 | 169 ± 81 |
| general content | 1236 ± 146 | 856 ± 108 | 552 ± 51 | 860 ± 207 | 455 ± 54 | 546 ± 265 |

Wheat sowing in sand pretreated with Rhodococcus qingshengii VKPM Ac-2143 strain after 14 days after bacterization led to the formation of more developed roots and shoots in wheat plants. Thus, at a concentration of the drug Pivot 28.5×10⁻⁷ ml/g of sand, the addition of microorganisms to the sand led to a 1.5-fold elongation of the roots (by 45%), at a concentration of this drug 114×10⁻⁷ ml/g of sand, a 1.2-fold elongation of the roots was observed (by 19%).

At the same time, in both cases, the length of shoots increased by more than 2 times compared to non-bacterized variants. At the same time, sowing wheat 21 days after the bacterization of sand led to a more pronounced effect of microorganisms on plant germination. In this case, at pesticide concentrations of 28.5×10⁻⁷ ml/g of sand and 114×10⁻⁷ ml/g of sand, an increase in root length was noted by 1.8 times (by 84%) and 1.5 times (by 49%), respectively. Also, in the sand treated with microorganisms, an increase in the length of the formed wheat shoots was noted by more than 3 times compared to plants sown in polluted soil without the addition of Rhodococcus qingshengii VKPM Ac-2143.

Thus, it was shown that the Rhodococcus qingshengii VKPM Ac-2143 strain has a positive effect on the morphological and biochemical characteristics of soft spring wheat plants.

1. Rhodococcus qingshengii bacterial strain, deposited under VKPM number Ac-2143, destructor of imazethapyr herbicide and growth stimulator of plants grown in soil contaminated with imazethapyr.
